(19) United States
(12) Patent Application Publication
(10) Pub. No.: US 2019/0184060 A1
(43) Pub. Date: Jun. 20, 2019

(54) EXTRACELLULAR MATRIX FOR TISSUE RECONSTRUCTION OF MUCOSAL TISSUE

(71) Applicant: Regentys Corporation, Miami Lakes, FL (US)

(72) Inventors: Richard Bulman, Coral Gables, FL (US); Christine Sapan, Miami Lakes, FL (US); Scott Winston, Boulder, CO (US)

(73) Assignee: Regentys Corporation, Miami Lakes, FL (US)

(21) Appl. No.: 16/326,252

(22) PCT Filed: Aug. 18, 2017
(86) PCT No.: PCT/US2017/047666

§ 371 Date: Feb. 18, 2019

Related U.S. Application Data
(60) Provisional application No. 62/377,173, filed on Aug. 19, 2016.

(51) Int. Cl. A61L 27/36; A61L 27/52; A61L 27/54; A61L 24/00

(52) U.S. Cl. CPC A61L 27/3633; A61L 27/52; A61L 27/3679; A61L 27/3629; A61L 27/54; A61L 2400/06; A61L 24/0015; A61L 24/0031; A61L 2430/22; A61L 2430/34; A61L 24/0005

(57) ABSTRACT

Described are methods and compositions for treatment of reproductive or urogenital mucosal tissue damage using Extracellular Matrix (ECM) solution and, particularly, Extracellular Matrix Hydrogel (ECMH) solution delivered to the target site for tissue repair, restoration, remodeling, or reconstruction.

### TECHNICAL FIELD OF THE INVENTION

The invention is directed to a method for using an extracellular matrix (ECM) or an extracellular matrix hydrogel (ECMH) topically administered to, or implanted in, an internal or external anatomic site in a patient in need thereof, to facilitate constructive remodeling at the site of diseased, damaged or missing tissue, particularly for restoration of mucosal tissue, and more particularly urogenital mucosal tissue, and thereby heal or functionally restore the diseased, damaged, or missing tissue.

### BACKGROUND

Damage to mucosal tissue can result from disease, chemical or radiation therapy, or trauma. The current trend towards minimally invasive, outpatient-based surgical procedures has prompted the development of injectable scaffolds, which can be inductive and bioactive or they can be non-inductive “place holders.” Purified collagen, gelatin, autologous fat, hyaluronic acid, and synthetic materials are clinically used as injectable scaffolds in regenerative medicine. However, overly-purified, chemically modified or synthetic materials can lead to adverse immune responses by the host and limit cell migration into the matrix.

Scaffolds composed of naturally occurring extracellular matrix (ECM) possess many bioactive properties that have been shown to lead to constructive remodeling of a variety of tissue types with minimization of scar tissue formation. ECM-derived scaffolds can be derived, for example, from the urinary bladder (urinary bladder matrix, or UBM) or small intestine (small intestinal submucosa, or SIS) of pigs or other animals, such as ruminants, and have been described for use for the repair of a variety of tissues. However, most current forms of ECM are limited by the material and geometrical properties inherent to the tissue from which they are derived (such as sheets or tubes of tissue) and delivery via injection is limited to powder suspensions.

One particularly useful ECM is a solubilized gel form, known as Extracellular Matrix Hydrogel (ECMH), which can be prepared using a method described in U.S. Pat. Nos. 8,361,503 and 8,691,276 and their progeny. The ECM or ECMH can be a sterilized soluble gel, prepared according to the method described in WO 2015/143310. These gels are advantageously fluid or liquid at room temperature and form a gel when heated above 25° C., for example, when coming into contact with the body of a patient having a normal body temperature which is about 37° C. Each of these patents and published patent applications is incorporated herein by reference in its entirety.

ECM and ECMH are useful for providing site-specific tissue repair of various tissues and are generally understood to have anti-microbial as well as regenerative properties. However, tissue remodeling or reconstruction using ECM or ECMH is dependent on a number of factors and successful treatment using ECM or ECMH for reconstruction of a particular tissue is highly unpredictable. The subject invention is directed to the unmet need of treating damaged or diseased mucosal tissues that have heretofore been difficult or resistant to treatment.

Thus, the subject invention relates to methods which provide treatment of particular conditions previously unknown to be treatable using ECM or ECMH, and thereby fulfilling an unmet need in the field of medical treatments for facilitating remodeling or healing of damaged or diseased mucosal tissue.

### SUMMARY OF THE INVENTION

The subject invention concerns a method for treatment of mucosal tissue damage using Extracellular Matrix (ECM) and particularly relates to treating damaged, diseased, or missing mucosal tissue using Extracellular Matrix Hydrogel (ECMH) delivered to the target site. As used herein, ECM means a devitalized extracellular matrix material derived from the extracellular matrix of one or more layers of an epithelial tissue. In a preferred embodiment, the ECMH is a solubilized homogeneous composition. In another preferred embodiment, the ECMH is a sterile, solubilized homogeneous composition. For purposes of clarification and distinction, a solubilized homogeneous composition is referred to as an “ECMH solution” and a sterile, solubilized homogeneous composition is referred to an “ECMH sterile solution.”

A gel ECMH solution or ECMH sterile solution can be prepared by a method comprising:
i) comminuting an ECM,
ii) solubilizing intact, non-dialyzed or non-cross-linked ECM by digestion with an acid protease in an acidic solution to produce a digest solution, and
iii) adjusting the pH of the digest solution to a pH between 7.2 and 7.8 to produce a neutralized digest solution.

An ECMH solution or ECMH sterile solution prepared by the above method can advantageously have the property of being a fluid or liquid solution at room temperature, e.g., 25° C., and forming a gel at a temperature greater than 25° C., and typically greater than 30° C.

In another embodiment, the method of preparing an ECM graft or scaffold composition further includes ultrasonicating the scaffold.

In carrying out a method of the invention, a scaffold or graft comprising ECMH solution or ECMH sterile solution is prepared as described herein, then implanted, transplanted, or administered to at least a section or area of tissue which is diseased or damaged, and allowed to remain in contact with the diseased or damaged tissue for a sufficient period of time so that replacement cells grow and replace the diseased or damaged cells. A sufficient period of time can be from one day to about six months.

The ECMH solution or ECMH sterile solution is a fluid composition which can advantageously be administered by injecting, infusing, spraying, or the like, to the site being treated. Such administration can be repeated several times, including one or more times a day for a period of several days, weeks or months, up to about one year as determined by the treating physician monitoring for acceptable regrowth or replacement of viable or healthy tissue, as desired.

One preferred embodiment of a sprayable gel, solution or suspension of a fluidized ECM or ECMH solution or sterile solution is to aerosolize the composition for administration by spraying. The aerosolized spray can be pumped from a source reservoir, through a cannula or other conduit provided in or with an endoscope manufactured or modified to deliver the fluidized ECM or ECMH composition to the desired site or location.

A method of the subject invention comprises administering an effective amount of ECM solution or ECMH sterile solution to a target site of mucosal tissue which is damaged or traumatized and would benefit from repair of the damaged or traumatized tissue by tissue reconstruction activated by the ECM composition.

Treatment of traumatized tissue can include mucosal or non-mucosal sites whereby ECM or ECMH is applied or otherwise administered to an area traumatized by a surgical procedure, e.g., tissue excision or incision, wherein the ECM or can be applied to the excised or incised site following the surgical procedure to facilitate healing and constructive remodeling of the tissue. Advantageously, the ECM or ECMH applied to the traumatized tissue can facilitate tissue repair with minimal or reduced scarring at the site.

Further, scar tissue already formed can be excised or otherwise removed e.g., by a surgical procedure performed by a physician, and the ECM or ECMH can be applied or otherwise administered to the newly formed wound such that the scar tissue is replaced by remodeled non-scar tissue facilitated by the ECM or ECMH.

Potential target mucosal tissue that can benefit from the subject method using ECMH or ECMH sterile solution according to the invention are oral mucosa; ocular mucosa; digestive tract mucosa, including esophagus, stomach, and intestinal tract; respiratory tract mucosa (nasal, lungs and trachea); reproductive tract mucosa; and genitourinary tract mucosa.

The method of the subject invention is particularly useful for treating damage or trauma to the female reproductive mucosa (e.g., cervical tissue) or female genitourinary tract mucosa (e.g., vaginal tissue) in view of the numerous potential causes of damage or trauma due to biopsy, STDs, rectovaginal fistula, vaginal prolapse, hormonal imbalance, PAP smears, sex, chemotherapy, radiation therapy, and the like. These tissues are routinely examined and easily accessible. ECM or ECMH can be useful to repair or restore function in tissue for conception, IVF or fertility treatments.

### DETAILED DESCRIPTION

The subject invention concerns remodeling, reconstruction or repair of damaged mucosal tissue by replacing or stimulating replacement of diseased or damaged mucosal cells and tissue in situ.

In one embodiment, a method of the invention comprises treating damaged or diseased mucosal tissue by stimulating stem cell or progenitor cell migration to the diseased or damaged mucosal tissue in a patient having symptoms of, or suffering from, disease or damage or trauma to mucosal lining of an internal organ.

More particularly, the subject invention comprises administering an ECMH solution or ECMH sterile solution to damaged or traumatized mucosal tissue by injection or infusion to the target site so that the stem cells or progenitor cells migrate to the target site and thereby stimulate or effect replacement or remodeling of damaged or diseased mucosal tissue with new, healthy mucosal tissue. Facilitation of constructive remodeling of tissue is referred to in the art as “tissue remodeling,” “tissue reconstruction,” “tissue restoration,” “tissue repair,” or “tissue regeneration”, and these terms are used interchangeably to refer to the process whereby healing of the tissue is facilitated by application, adherence, attachment or other conventional administration of an ECM or ECMH composition and results in tissue repair more effective or more efficient or less invasive than if healing were allowed to occur by natural processes or with the use of non-ECM or non-ECMH material or composition.

In another embodiment of the invention, stem cells or progenitor cells or an active drug can be incorporated into the ECMH solution or ECMH sterile solution composition, and that solution comprising stem cells, progenitor cells, or active drug, can be delivered to the target mucosal tissue by delivery means appropriate for the composition as described herein. For example, a fluid ECMH solution or ECMH sterile solution can be infused, injected, or sprayed onto the target tissue exhibiting damage or trauma.

By design, ECMH solution or ECMH sterile solution encourages an inflammatory response, including migration of macrophages to the site. One unique characteristic of ECMH solution or ECMH sterile solution is to encourage a switch in the phenotype of these macrophages from type M1 (pro-inflammatory) to type M2 (pro-tissue remodeling, such as would be seen in a person without an underlying pro-inflammatory condition). An important ramification of this M1 to M2 phenotype switch is to allow tissue that is otherwise predisposed to inflammation to remain healthy.

A fluidized ECM, such as the ECMH solution or ECMH sterile solution of the invention can be administered by a commonly employed fluid administration or delivery process such as injection, infusion or drench (“squirting” onto the site) or by spraying the fluid or aerosolized fluid onto the site of the damaged, diseased, or missing tissue in need of repair. A preferred spraying technique is carried out using an aerosolized ECM fluid, delivered, for example endoscopically. Alternatively, the fluidized ECM can be applied using an applicator or device which can facilitate its application, such as, in the case of uterine or vaginal or internal organ tissue repair, a tampon, tampon applicator, and the like.

As used herein, the terms “composition,” “material,” “scaffold,” and “graft,” as referring to ECM, can be used interchangeably, and do not connote a particular configuration, such as being in solid, liquid, fluid or other form. For example, an “ECM scaffold” can be solid or fluid, including a liquid or gel.

Preferably, a fluidized ECM composition, such as ECMH solution or ECMH sterile solution composition as described herein, forms a gel in situ such that it has viscous properties, and has a viscosity sufficient so that the composition adheres to the desired location within the body for a sufficient period of time to carry out its healing effect, either by a single administration or application, or by multiple or repeated administrations or applications.

In a preferred embodiment, the fluidized ECMH solution or ECMH sterile solution is a hydrogel having liquid or fluid properties before, and when, applied or administered, which advantageously thickens or becomes more viscous, forming an adhesive gel upon, or shortly following, contact with the body at the site of administration or application.

Gelation can be initiated and effected by increased temperature, such as body heat following administration. The fluid ECMH solution or ECMH sterile solution can also be mixed with a separate viscous agent, such as a hydrogel or other commonly known gelling agent to provide sufficient viscosity. Alternatively, two fluids can be administered whereby at least one of the fluids contains ECMH solution or ECMH sterile solution, wherein the two fluids gel when coming into contact with one another or when mixed.

It is well established that an ECM composition can cause stem cells to migrate to the site when sufficient vascularization of the tissue exists, and the blood vessels provide an adequate conduit for the cells to migrate to the site. In addition, ECM is understood to communicate with the adjacent or underlying tissue via chemical signaling, and the adjacent or underlying tissue communicates with the ECM, by a phenomenon termed “dynamic reciprocity” which optimizes cell and tissue remodeling, replacement or regeneration where ECM is used. Thus, the ECM composition can serve as a scaffold for new cellular and tissue growth at the site of placement, administration, or application.

In one embodiment, an ECM can be derived from tissue commonly used in the art. For example, ECM has been derived from small intestinal submucosa (SIS) or urinary bladder matrix (UBM) tissue. In an alternative embodiment, the ECMH solution or ECMH sterile solution can be derived from the same tissue type as the tissue being treated, referred to as “tissue-specific extracellular matrix” or “TS-ECM.” TS-ECM can provide advantageous results, such as more efficient or more responsive reconstructive tissue remodeling, which can occur through dynamic reciprocity. For purposes of describing the subject invention, reference to “ECM” in the context of use in accordance with the subject method means ECMH solution, ECMH sterile solution, or TS-ECM, wherein the TS-ECM is either TS-ECMH solution or TS-ECMH sterile solution.

Thus, the subject invention includes a method and composition for treatment of disease- or trauma-damaged mucosal tissue, such as reproductive, integumentary, pancreatic, renal, circulatory, urogenital, or respiratory tissue, by administering ECMH solution or ECMH sterile solution, or TS-ECM. The method of the subject invention can be useful in male or female tissue including male or female urogenital tissue.

In one embodiment, TS-ECM is derived from the same species as the species being treated. For example, treatment of UC in a human will employ human mucosal tissue as the TS-ECM composition. The tissue graft can be an allograft, as described above, or xenograft.

Advantageously, it is contemplated that a treatment or cure of damage or trauma to mucosal tissue can be effected by a method comprising implantation, transplantation or administration of ECMH solution, ECMH sterile solution, or TS-ECM without a prior step of mucosal resection or ablation. Thus, in such instances where disease or damage to the tissue creates a lesion, such as an exposed or bleeding surface of the tissue, resection of the tissue prior to may be omitted.

As used herein, the terms implanting or implantation, transplanting or transplantation, applying or application, administering or administration, infusing or infusion, injecting or injection, delivering or delivery, all refer to the process or providing ECMH solution, ECMH sterile solution, or TS-ECM to the site of treatment, and would be understood by a person of ordinary skill in the art to have the same meaning, depending on the composition properties and procedure employed for carrying out the delivery of ECM to the site. These terms can be used interchangeably and are in no way limiting to the method of the invention.

Non-limiting examples of sites and applications where the ECMH solution, ECMH sterile solution or TS-ECM according to the invention may be useful are lumpectomy sites, tumor removal sites, tissue voids due to trauma or tissue/organ excision, organ lesion locations, wounds, dental alveolus, thoracic cavity, abdominal cavity, pulmonary or hepatic lobectomy, vagina, urethra, uterus, pelvic cavity, urinary bladder, penis and penile tissue, subcutaneous tissue, nasal cavity or sinus, vessels, and intramedullary cavity.

In one embodiment of the invention, a spray is made from the ECMH solution, ECMH sterile solution, or TS-ECMH described above that is sprayed onto an anatomic site requiring tissue reconstruction. The ECMH is loaded into a delivery system that can be introduced by minimally invasive procedures. The final delivery of the ECMH is achieved by pressure or physical advancement of the ECMH material through a sheath, syringe, or nozzle such that the ECMH solution or ECMH sterile solution is applied at the desired site. Spray width and area can be controlled by the size of the opening of the sheath, syringe, or nozzle. Alternatively, a fluid ECM or ECMH can be applied directly to the site using an applicator, including a tampon applicator or tampon, itself, or other medical or personal hygiene applicator device.

Advantageously, an ECMH used in a method of the invention expands or polymerizes in vivo when injected into a site and occupying space. When the polymerizable ECMH solution or ECMH sterile solution is administered to a target site, it polymerizes and stays fixed in position to carry out its intended purpose of tissue reconstruction. ECMH solution or ECMH sterile solution can polymerize after a change in pH (higher to lower, or lower to higher) or temperature (preferably lower to higher). Once the polymerizable ECMH solution or ECMH sterile solution is introduced into the desired site, the pH at the site can be altered by an addition of an acid or base enabling the gel to polymerize in vivo at the application site. Polymerizable ECMH solution or ECMH sterile solution can also polymerize at body pH after application to the desired site.

Polymerizable ECMH solution can be sterilized by, for example, radiation (e-beam, gamma) or gas (ethylene oxide or nitric dioxide), or other sterilization procedure currently known in the art, such as supercritical CO₂ processes which are described.

### EXAMPLES

### Example 1—Application of ECMH Solution for Replacement of Mucosal Tissue in a Rat Model

In accordance with the invention, the feasibility of using an ECMH solution for treatment of vaginal mucosa can be tested in vivo in an animal model. Rats may be established as a model for lesions of the human female reproductive tract, such as vaginal mucosal tissue, whereby lesions can be chemically induced in rat vaginal tissue.

Treatment of the induced lesions can be carried out using a ECMH solution or ECMH sterile solution derived from small intestine submucosa (SIS) and introduced into to the site by injection or spray using a syringe, cannula, catheter, endoscope, colposcope, tampon or tampon applicator, or another suitable delivery device. The ECMH solution or ECMH sterile solution can be administered as a single administration or application or, alternatively, as multiple administrations or applications, e.g., as needed (prn), one or more times-per-day for a period of several days up to about one week, up to about one month or more as determined by a treating physician or other healthcare professional. One preferred dosing regimen for ECMH solution or ECMH sterile solution can be once per day for 7-30 days.

### Method of Preparation of Gels from ECM

The preparation of SIS from a segment of small intestine is detailed in U.S. Pat. Nos. 4,902,508, 5,275,826, and 5,514,533, the disclosures of which are expressly incorporated herein by reference. A segment of intestine is first subjected to abrasion using a longitudinal wiping motion to remove both the outer layers (particularly the tunica serosa and the tunica muscularis) and the inner layers (the luminal portions of the tunica mucosa). Typically, the SIS is rinsed with saline and optionally stored in a hydrated or dehydrated state until use as described below.

The present fluidized compositions are prepared as solutions of intestinal submucosa by comminuting and/or digesting the submucosa with a protease, such as trypsin or pepsin, for a period of time sufficient to solubilize said tissue and form a substantially homogeneous solution. The intestinal submucosa starting material is comminuted by tearing, cutting, grinding, shearing and the like. Grinding the submucosa in a frozen or freeze-dried state is preferred although good results can be obtained as well by subjecting a suspension of pieces of the submucosa to treatment in a high speed (high shear) blender and dewatering, if necessary, by centrifuging and decanting excess water. The comminuted intestinal submucosa can be dried to form a submucosa powder. Thereafter, it can be hydrated, that is, combined with water or buffered saline and optionally other pharmaceutically acceptable excipients to form ECMH solution as a fluid having a viscosity of about 2 to about 300,000 cps at 25° C. The higher viscosity graft compositions can have a gel or paste consistency. The present compositions can be sterilized using art-recognized sterilization techniques such as exposure to ionizing radiation or sterilizing gas, e.g., supercritical CO₂.

The fluidized submucosa of this invention also finds use as an injectable heterograft for tissues, for example, soft tissues, in need of repair or augmentation most typically to correct trauma or disease-induced tissue defects.

### SIS Solution

SIS powder is sifted through a wire mesh into any convenient vessel. The powder is then subjected to proteolytic digestion to form a substantially homogeneous solution. In one embodiment, the powder is digested with 1 mg/ml of pepsin (Sigma Chemical Co., St. Louis, Mo.) in 0.1 M acetic acid, adjusted to pH 2.5 with HCl, over a 48-hour period at room temperature. The reaction medium is neutralized with sodium hydroxide to inactivate the peptic activity. The solubilized submucosa may then be concentrated by salt precipitation of the solution and separated for further purification and/or freeze drying to form a protease solubilized intestinal submucosa in powder form.

The viscosity of fluidized submucosa compositions in accordance with this invention can be manipulated by controlling the concentration of the submucosa component and the degree of hydration. The viscosity can be adjusted to a range of about 2 to about 300,000 cps at 25° C. Low viscosity submucosa compositions are better adapted for intra-articular applications or applications within body cavities. Higher viscosity formulations, for example, gels, can be prepared from the SIS digest solutions by adjusting the pH of such solutions to about 6.0 to about 7.0. Gel forms of the present compositions, as submucosa suspensions or submucosa digest solutions, are typically preferred for mucosal, subcutaneous or intramuscular applications using syringes or catheters.

SIS gel has also been described as being formed into a gel by mixing 0.1 N NaOH ( 1/10 of the volume of digest solution) and 10×PBS pH 7.4 ( 1/9 of the volume of digest solution) in appropriate amounts at 4° C. The solution was brought to the desired volume and concentration using cold (4° C.) 1×PBS pH 7.4 and placed in a 37° C. incubator for gelation to occur.

The ECM was able to form a matrix after 40 minutes in solution. The ECM-derived gel was liquid at temperatures below 20° C. but turns into a gel when the temperature is raised to 37° C.

In preparing gels from ECM, all of the solutions should be kept on ice and the following variables must be determined in accordance with U.S. Pat. No. 8,361,503, which is hereby incorporated by reference in its entirety:
C_{f}=concentration of the final gel in mg/ml
Cₛ=concentration of the ECM digest solution in mg/ml
V_{f}=volume of the final gel solution needed for the experiments
V_{d}=volume needed from the ECM digest solution in ml
V_{10X}=volume of 10×PBS needed in ml
V_{1X}=volume of 1×PBS needed in ml
V_{NaOH}=volume of 0.1 N NaOH needed in ml

First, determine the final concentration (C_{f}) and volume (V_{f}) of ECM gel required. Then, calculate the mass of ECM needed by multiplying C_{f}(mg/ml)×V_{f}(ml). This value will give you the volume needed from the ECM digest solution (V_{d}), where V_{d}=[C_{f}(mg/ml)×V_{f}(ml)]/Cₛ.

Calculate the volume of 10×PBS needed by dividing the calculated volume V_{d} by 9 (V_{10X}=V_{d}/9). Calculate the volume of 0.1 N NaOH needed by dividing the calculated volume V_{d} by 10 (V_{NaOH}=V_{d}/10). Calculate the amount of 1×PBS needed to bring the solution to the appropriate concentration/volume as follow: V_{1X}=V_{f}−V_{d}−V_{10X}−V_{NaOH}. Add all the reagent (V_{1X}+V_{d}+V_{10X}+V_{NaOH}) to an appropriate container (usually 15 or 50 ml centrifuge tubes) without the ECM digest (V_{d}). Place solutions on ice and keep on ice at all times.

Add the appropriate volume from the ECM digest solution (V_{d}) to the PBS/NaOH mixture prepared above and mix well with a 1 ml micropipette while being careful and avoiding the creation of air bubbles in the solution. Depending on the viscosity of the ECM digest solution, there might be some significant volume loss during the transfer. Monitor the total volume and add appropriate amounts until the final volume is achieved. Measure the pH of the pre-gel solution, where pH should be around 7.4.

Add the pre-gel solution to a mold or to appropriate wells. Place the mold or wells in a 37° C. incubator for a minimum of 40 minutes. Avoid using an incubator with CO₂ control. If water evaporation is a concern, place the mold inside a plastic zip-lock bag before placing in the incubator. After gelation, the gel can be removed from the mold and placed on 1×PBS. If the gels were made in tissue culture plates, 1×PBS can be placed on top of the gels until use to maintain the gels hydrated. Sample calculation: Make 6 ml of gel with a final concentration of 6 mg/ml from the 10 mg/ml stock solution.

### SIS-ECM Administration Procedure

SIS-ECM solution can be administered by syringe into the vagina of the lesion-induced rats. No rejection, infection, or abnormal physiologic response of the host animal is expected following administration of the graft. The solution may also be administered via endoscopy or via laparoscopy into the vagina, or may be administered using a tampon or tampon applicator or similar device. It is believed that an unexpected result of the current invention will be stimulation of appropriate tissue remodeling such that augmentation of vaginal mucosa can be accomplished with ECMH solution derived from SIS.

The fluidized compositions of this invention can result in tissue replacement and repair, and further result in treatment or cure of lesions caused by disease of the mucosal tissue. The ECMH solution is used in accordance with the present method to induce regrowth of natural mucosal tissue. By applying an effective amount of ECMH solution into the locale of the defective tissue, the biotropic properties can be realized without the need for more invasive surgical techniques.

Variations, modifications, and other implementations of what is described herein will occur to those of ordinary skill in the art without departing from the spirit and scope of the invention as claimed. Accordingly, the invention is to be defined not by the preceding illustrative description but rather by the spirit and scope of the following claims.

Each of the references provided herein are incorporated by reference in their entirety.

### Example 2—Gel ECM for Repair of Mucosal Tissue Such as Vaginal Mucosa

In accordance with the invention, the feasibility of using an ECM gel composition for inducing restoration, remodeling, or repair of a mucosal tissue can be tested in vivo in rabbits. Rabbits are established as a model for human vaginal irritation, inflammation, ulceration, epithelial disruption and edema.

Irritation, inflammation, ulceration, epithelial disruption, and edema of the vaginal mucosa can be induced in rabbits by directly applying microbicides such as benzalkonium chloride (BZK), acid solutions, liquid nitrogen, or chemotherapeutics onto the vaginal mucosal surface.

Treatment of damaged vaginal mucosal tissue can be carried out using a gel extracellular matrix (ECM) derived from small intestine submucosa (SIS) and introduced onto the vaginal mucosal surface by injection or spray using a syringe, cannula, catheter, endoscope, colposcope, tampon, applicator stick or other suitable delivery device. The gel ECM can be administered one or more times-per-day for a period of at least one week, and up to about one month. A preferred dosing regimen for gel ECM is once per day for 1-5days.

### Method of Preparation of Gels from ECM

The preparation of gel ECM from SIS is described in Example 1. The gel ECM solution will be viscous at temperatures less than 25° C., but will gel at 37° C. within 10-30 minutes and preferably within 20 minutes.

### In Vivo Study Using Gel ECM in a Rabbit Model of Vaginal Mucosal Tissue Damage

An approach to treating vaginal irritation, inflammation, ulceration, epithelial disruption and edema is to provide an ECM composition that can provide a physical barrier to irritants, microorganisms or inflammatory cellular responses and provide a scaffold for rapid replacement of the vaginal mucosal barrier function by restoring mucosal epithelial structure and organizing the endogenous mucosal healing processes. This study will demonstrate efficacy of local delivery of a hydrogel form of mammalian extracellular matrix (ECMH) for treating damaged vaginal mucosal tissue. The effect of ECMH on clinical symptomology, inflammation, and epithelial barrier function will be evaluated by multiple outcome measures.

The study, presented in this Example, will show that a hydrogel composed of ECM can effectively treat a rabbit model of vaginal mucosal tissue irritation. Effective therapy is determined according to two essential physiologic processes that can be positively directed by ECMH treatment: 1) restoration of vaginal epithelial barrier function, which protects the host from the relentless barrage of pro-inflammatory luminal contents and microorganisms; and 2) healing of the damaged vaginal mucosal tissue more rapidly and with less scarring than no treatment. This study can demonstrate effective treatment of vaginal mucosal tissue damage with ECMH by restoring epithelial barrier function, and establishing the environment for mucosal tissue repair and regeneration.

### Methods

### Experimental Design

To determine the efficacy of ECMH in a rabbit model of vaginal tissue irritation (RVI), mucosal tissue damage can be induced in female rabbits by applying a 1 mL solution of 2% benzalkonium chloride (BZK) directly in the vagina for 10 consecutive days with a syringe and cannula inserted 5-6 inches into the vagina (Fields et al. 2014). ECMH may be prepared as described in Example 1. Either 1 mL of an ECMH or Control solution mimicking the ECMH diluent including processing residuals will be applied with a syringe and cannula placed 5-6 inches into the vagina once daily for five days. Animals will be sacrificed at 7 days and 14 days post BZK application to evaluate the temporal response (n=14 per time point per treatment).

Healthy rabbits, which did not receive any BZK or treatment, will be included for comparison at both 7 and 14 days (n=6 per time point). The primary study endpoints may include clinical response, histologic scores, inflammation response, and barrier function.

All in-vivo studies will use an ECMH concentration 6-10 mg/mL, preferably 8 mg/mL and all in-vitro studies will use an ECMH concentration of 400-600 μg/mL, preferably 500 μg/mL. For studies visualizing the binding of ECMH to vaginal mucosal tissue, FITC-labeled ECMH can be prepared with a protein labeling kit per manufacturer's instructions (Thermo PierceNet; #53027).

### Animals and Husbandry

All procedures and animal studies will be approved and conducted in compliance with all applicable regulations on the care and testing of research animals. Female rabbits, 20-28 weeks of age, will be obtained and individually housed and environmentally acclimated for 7-10 days. Animals will be housed under standard conditions with a temperature of 21-23° C. and 12 hr dark/light cycles. Rabbits will be allowed ad libitum access to food and water throughout the study.

### Macroscopic Examination

Rabbits will be examined daily for signs of vaginal discharge or edema.

### Explanting and Scoring Vaginal Tissue

Animals will be sacrificed at determined time points as described previously. Euthanasia will be achieved by CO2 inhalation and subsequent cervical dislocation in accordance with the American Veterinary Medical Association (AVMA). Following euthanasia vaginal tissue samples will be obtained from the cervix, central vagina, and caudal vagina.

Tissue sections will be used for histology and myeloperoxidase measurement. The sections will be paraffin embedded and sections (5 μm) will be obtained from the cervix, central vagina, and caudal vagina, and stained with hematoxylin and eosin (H&E) for representative histologic scoring. Sections will be scored for leukocyte infiltration, epithelial ulceration and disruption, and edema.

### In Vitro Testing of ECMH for Adherence and Barrier Function

### ECMH Adhesion Testing

The mucoadhesion of ECMH can be measured using a modified detachment force measurement. A uniaxial tensile testing machine (MTS Insight; MTS Systems Corp., Eden Prairie, Minn.) equipped with a 10N load cell will be used for all tensile strength measurements. Two rabbit vaginal tissue sections can be glued to steel washers (diameter 12.7 mm) with mucosa facing outward and one washer glued to the bottom of a 24-well plate (diameter 15.6 mm). The ECMH can be prepared by neutralizing with one tenth volume of 0.1M NaOH followed by the addition of one ninth volume of 10×PBS. Then 0.5 mL of ECMH can be added onto both the bottom washer and the top washer and allowed to penetrate into the gel to a predetermined depth before incubating at 37° C. for 1 hour. After incubation, the mucosa can be slowly withdrawn upwards at a constant speed of 5 mm/min until a failure occurs between the surfaces. (Chickering & Mathiowitz (1995); (Kammer 1983).

### ECMH Retention Studies with FITC

To determine hydrogel retention time, nine rabbits will be administered FITC-labeled ECMH following tissue damage induction with BZK, and will be sacrificed at 2 hr, 12 hr, and 24 hr post enema (n=3 per time point). Explanted vaginal tissue from FITC-ECMH treated rabbits will processed to be optically clear such that the luminal contents will be visible by fluorescent imaging. Immediately following sacrifice all samples will be protected from light to prevent photo bleaching of the FITC conjugate. Optical clearing of the tissue can be initiated by incubating in Dent's fixative (1:4 dimethyl sulfoxide (DMSO):acetone) for 2 hours. Vaginal tissue can then be permeabilized and bleached in Dent's bleach (1:4:1 DMSO:acetone:H2O2) for 1 hour. Optically cleared tissue will then be imaged. Exposure time will be set to a control sample of FITC-ECMH and kept constant for all subsequent images.

### TRITC-Dextran Permeability Assay

Vaginal mucosal epithelial permeability will be assessed by intravaginal administration of TRITC-dextran (molecular mass 4.4 kDa; Sigma). Rabbits will be administered TRITC-dextran (1 mL, 10 mg/mL) 4 h before sacrifice. Whole blood from cardiac juncture will be obtained at the time of sacrifice and collected in serum tubes. TRITC-dextran measurements will be performed in triplicate on a SpectraMax plate reader (Molecular Devices), with serial dilutions of TRITC-dextran used as a standard curve.

### Vaginal Epithelial Cell (VEC) cCulture

For in-vitro barrier function assays, VEC (VK2/E6E7, ATCC) will be cultured to about 80% confluence in MEM containing non-essential amino acids, 1 mM sodium pyruvate, and 20% FBS, The functional response of VEC to ECMH will be evaluated using rapid differentiation system (Corning Biocoat HTS) per manufacturer's instructions. Confluent and differentiated cell monolayers will be challenged with 100 ng/mL LPS for 2 hours and then treated with ECMH for 48 hours. The functional response of VEC will be measured by transepithelial electrical resistance (TEER) and the presence of epithelial cadherin (E-cadherin) cell adhesion protein.

Overall, the test results can demonstrate that ECMH adheres to the vaginal mucosa, that it provides a physical barrier by coating the vaginal mucosa, restores the epithelial barrier function, and acts as a scaffold for the process of tissue repair. The fluidized compositions of this invention may result in tissue replacement and repair, and further result in treatment or cure of lesions caused by disease of the mucosal tissue. The ECMH solution can be used in accordance with the present method to induce regrowth of natural mucosal tissue. By applying an effective amount of ECMH solution into the locale of the defective tissue, the biotropic properties can be realized without the need for more invasive techniques or systemic pharmaceutical treatments.

### Example 3—Gel ECM for Repair of Mucosal Tissue Such as Vaginal Mucosa

In accordance with the invention, the feasibility of using an ECM gel composition for inducing restoration, remodeling, or repair of a mucosal tissue can be tested in vivo in guinea pig. Guinea pigs are established as a model for human vaginal irritation, inflammation, ulceration, epithelial disruption and edema.

Irritation, inflammation, ulceration, epithelial disruption, and edema of the vaginal mucosa can be induced in guinea pigs by infection with herpes simplex virus type 2 (HSV2) (Mayo and Hsiung, 1984)

Treatment of damaged vaginal mucosal tissue can be carried out using a gel extracellular matrix (ECM) derived from small intestine submucosa (SIS) and introduced onto the vaginal mucosal surface by injection or spray using a syringe, cannula, catheter, endoscope, colposcope, tampon, applicator stick or other suitable delivery device. The gel ECM can be administered one or more times-per-day for a period of at least one week, and up to about one month. A preferred dosing regimen for gel ECM is once per day for 1-5 days.

### Method of Preparation of Gels from ECM

The preparation of gel ECM from SIS is described in Example 1. The gel ECM solution will be viscous at temperatures less than 25° C., but will gel at 37° C. within 10-30 minutes and preferably within 20 minutes.

### In Vivo Study Using Gel ECM in a Guinea Pig HSV 2 Model of Vaginal Mucosal Tissue Damage

An approach to treating vaginal irritation, inflammation, ulceration, epithelial disruption and edema is to provide an ECM composition that can provide a physical barrier to irritants, microorganisms or inflammatory cellular responses and provide a scaffold for rapid replacement of the vaginal mucosal barrier function by restoring mucosal epithelial structure and organizing the endogenous mucosal healing processes. This study will demonstrate efficacy of local delivery of a hydrogel form of mammalian extracellular matrix (ECMH) for treating damaged vaginal mucosal tissue. The effect of ECMH on clinical symptomology, inflammation, and epithelial barrier function will be evaluated by multiple outcome measures.

The study, presented in this Example, will show that a hydrogel composed of ECM can effectively treat a guinea pig model of vaginal mucosal tissue irritation. Effective therapy is determined according to two essential physiologic processes that can be positively directed by ECMH treatment: 1) restoration of vaginal epithelial barrier function, which protects the host from the relentless barrage of pro-inflammatory luminal contents and microorganisms; and 2) healing of the damaged vaginal mucosal tissue more rapidly and with less scarring than no treatment. This study can demonstrate effective treatment of vaginal mucosal tissue damage with ECMH by restoring epithelial barrier function, and establishing the environment for mucosal tissue repair and regeneration.

### Methods

### Experimental Design

To determine the efficacy of ECMH in a guinea pig HSV 2 model, mucosal tissue damage can be induced in female guinea pigs with a 1 mL inoculum containing 104.5 to 105 particle forming units of HSV 2 placed directly in the vagina with a tuberculin syringe without a needle after which the vagina is plugged with a surgical pad. ECMH may be prepared as described in Example 1. Either 1 mL of an ECMH or Control solution mimicking the ECMH diluent including processing residuals will be applied with a syringe and cannula placed into the vagina and on the vulva once daily for five days, beginning 24-72 hours after virus inoculation. Animals will be sacrificed at 3 days, 7 days, and 14 days post treatment to evaluate the temporal response (n=14 per time point per treatment). Healthy guinea pigs, which did not receive any virus or treatment, will be included for comparison at both 7 and 14 days (n=6 per time point). The primary study endpoints may include clinical response, histologic scores, inflammation response, and barrier function.

All in-vivo studies will use an ECMH concentration 6-10 mg/mL, preferably 8 mg/mL. For studies visualizing the binding of ECMH to vaginal mucosal tissue, FITC-labeled ECMH can be prepared with a protein labeling kit per manufacturer's instructions (Thermo PierceNet; #53027).

### Animals and Husbandry

All procedures and animal studies will be approved and conducted in compliance with all applicable regulations on the care and testing of research animals. Young, adult female Harley guinea pigs, will be obtained and individually housed and environmentally acclimated for 7-10 days. Animals will be housed under standard conditions with a temperature of 21-23° C. and 12 hr dark/light cycles. Guinea pigs will be allowed ad libitum access to food and water throughout the study.

### Macroscopic Examination

Guinea pigs will be examined daily for signs of vaginal discharge or edema.

### Explanting and Scoring of Vaginal Tissue

Animals will be sacrificed at determined time points as described previously. Euthanasia will be achieved by CO2 inhalation and subsequent cervical dislocation in accordance with the American Veterinary Medical Association (AVMA). Following euthanasia vaginal tissue samples will be obtained from the cervix, central vagina, caudal vagina and vulva.

Tissue sections will be used for histology and myeloperoxidase measurement. The sections will be paraffin embedded and sections (5 μmm) will be obtained from the cervix, central vagina, caudal vagina and vulva and stained with hematoxylin and eosin (H&E) for representative histologic scoring. Sections will be scored for leukocyte infiltration, epithelial ulceration and disruption, and edema.

### Virus Quantitation from Vaginal Swabs

Shedding of virus will be measured by either swabbing the vagina of each guinea pig with a cotton-tipped, premoistened swab on Day 3, Day 7, and Day 10 post initiation of treatment or by obtaining a cervical vaginal lavage on Day 3, Day 7, and Day 10 post initiation of treatment. The quantity of HSV 2 in the secretions will be measured by real time PCR.

### In Vitro Testing of ECMH for Adherence and Barrier Function

### Methods

### ECMH Adhesion Testing

The mucoadhesion of ECMH can be measured using a modified detachment force measurement. A uniaxial tensile testing machine (MTS Insight; MTS Systems Corp., Eden Prairie, Minn.) equipped with a 10N load cell will be used for all tensile strength measurements. Two guinea pig vaginal tissue sections can be glued to steel washers (diameter 12.7 mm) with mucosa facing outward and one washer glued to the bottom of a 24-well plate (diameter 15.6 mm). The ECMH can be prepared by neutralizing with one tenth volume of 0.1M NaOH followed by the addition of one ninth volume of 10×PBS. Then 0.5 mL of ECMH can be added onto both the bottom washer and the top washer and allowed to penetrate into the gel to a predetermined depth before incubating at 37° C. for 1 hour. After incubation, the mucosa can be slowly withdrawn upwards at a constant speed of 5 mm/min until a failure occurs between the surfaces. (Chickering & Mathiowitz (1995); (Kammer 1983).

### ECMH Retention Studies with FITC

To determine hydrogel retention time, nine guinea pigs will be administered FITC-labeled ECMH 3-5 days post viral inoculation. and will be sacrificed at 2 hr, 12 hr, and 24 hr post ECMH application (n=3 per time point). Explanted vaginal tissue from FITC-ECMH treated guinea pigs will processed to be optically clear such that the luminal contents will be visible by fluorescent imaging. Immediately following sacrifice all samples will be protected from light to prevent photo bleaching of the FITC conjugate. Optical clearing of the tissue can be initiated by incubating in Dent's fixative (1:4 dimethyl sulfoxide (DMSO):acetone) for 2 hours. Vaginal tissue can then be permeabilized and bleached in Dent's bleach (1:4:1 DMSO:acetone:H2O2) for 1 hour. Optically cleared tissue will then be imaged. Exposure time will be set to a control sample of FITC-ECMH and kept constant for all subsequent images.

### TRITC-Dextran Permeability Assay

Vaginal mucosal epithelial permeability will be assessed by intravaginal administration of TRITC-dextran (molecular mass 4.4 kDa; Sigma). Guinea pigs will be administered TRITC-dextran (1 mL, 10 mg/mL) 4 h before sacrifice. Whole blood from cardiac juncture will be obtained at the time of sacrifice and collected in serum tubes. TRITC-dextran measurements will be performed in triplicate on a SpectraMax plate reader (Molecular Devices), with serial dilutions of TRITC-dextran used as a standard curve.

Overall, the test results can demonstrate that ECMH adheres to the vaginal mucosa, that it provides a physical barrier by coating the vaginal mucosa, restores the epithelial barrier function, and acts as a scaffold for the process of tissue repair. The fluidized compositions of this invention may result in tissue replacement and repair, and further result in treatment or cure of lesions caused by disease of the mucosal tissue. The ECMH solution can be used in accordance with the present method to induce regrowth of natural mucosal tissue. By applying an effective amount of ECMH solution into the locale of the defective tissue, the biotropic properties can be realized without the need for more invasive techniques or systemic pharmaceutical treatments.

### Example 4—Anti-Scarring Use

A patient presenting with a scar from prior trauma or surgical procedure can be treated with ECM or ECMH. A physician or surgeon excises the scar tissue and any margin thereof as determined appropriate by the physician or surgeon. ECM or ECMH is applied to the tissue edges surrounding the excised scar tissue. Closure of the wound formed by excision of the scar and surrounding tissue is performed by conventional or standard wound closure procedure (e.g., suture or adhesive.) ECM or ECMH can alternatively or further be applied to the closed wound tissue. Further or repeated application of ECM or ECMH can be performed as determined for facilitating constructive remodeling of the tissue without scar formation or with reduced scar formation.

Alternatively, a surgical procedure is performed on a patient where the surgical site does not have scar tissue already formed. ECM or ECMH can be applied to the incision or excision site immediately after the procedure and prior to closure of the surgical site by suture or adhesive. Additional ECM or ECMH material can be applied to the closed surgical site to facilitate constructive remodeling of the tissue and prevent or reduce scar tissue formation.

The subject invention described herein is not limited by the description or examples provided, and is intended to encompass embodiments which enable a person of ordinary skill in the art to practice the invention falling within the scope and spirit of the invention as described.

1. A composition for inducing restoration, remodeling, or repair of reproductive or urogenital mucosal tissue in a mammal, said composition comprising an extracellular matrix hydrogel composition selected from ECMH solution, ECMH sterile solution and TS-ECMH administered in an effective amount administered in an effective amount to damaged or traumatized reproductive or urogenital tract mucosal tissue in need of repair, and facilitating constructive tissue remodeling or repair of the damaged or traumatized reproductive or urogenital tract mucosal tissue.

2. The composition of claim 1 wherein the extracellular matrix is derived from small intestine submucosa (SIS).

3. The composition of claim 1 wherein the extracellular matrix is derived from urinary bladder mucosa (UBM).

4. The composition of claim 1 wherein the extracellular matrix is a tissue-specific extracellular matrix (TS-EMC).

5. The composition of claim 1 wherein said mucosal tissue is vaginal tissue.

6. The composition of claim 1 wherein said mucosal tissue is external genitalia tissue.

7. The composition of claim 1 wherein said mucosal tissue is uterine tissue.

8. The composition of claim 1 wherein said mucosal tissue is cervical tissue.

9. A method for inducing restoration, remodeling, or repair of reproductive or urogenital tract mucosal tissue in a mammal, said method comprising:
providing an extracellular matrix hydrogel composition selected from ECMH solution, ECMH sterile solution and TS-ECMH;
administering an effective amount of the extracellular matrix hydrogel to damaged or traumatized reproductive or urogenital tract mucosal tissue in need of repair;
thereby facilitating constructive tissue remodeling or repair of the damaged or traumatized reproductive or urogenital tract mucosal tissue.

10. The method of claim 9 wherein the extracellular matrix is derived from small intestine submucosa (SIS).

11. The method of claim 9 wherein the extracellular matrix is derived from urinary bladder mucosa (UBM).

12. The method of claim 9 wherein the extracellular matrix is a tissue-specific extracellular matrix (TS-EMC).

13. The method of claim 9 wherein said reproductive or urogenital tract mucosal tissue is vaginal tissue.

14. The method of claim 9 wherein said reproductive or urogenital tract mucosal tissue is external genitalia tissue.

15. The method of claim 9 wherein said reproductive or urogenital tract mucosal tissue is uterine tissue.

16. The method of claim 9 wherein said reproductive or urogenital tract mucosal tissue is cervical tissue.

17. A method for inducing restoration, remodeling, or repair of reproductive or urogenital tract mucosal tissue in a mammal, said method comprising:
providing an extracellular matrix hydrogel composition selected from ECMH solution, ECMH sterile solution and TS-ECMH;
administering an effective amount of the extracellular matrix hydrogel to damaged or traumatized reproductive or urogenital tract mucosal tissue in need of repair;
thereby facilitating constructive tissue remodeling or repair of the damaged or traumatized reproductive or urogenital tract mucosal tissue with limited or reduced formation of scar tissue.

18. The method of claim 17 wherein the tissue is traumatized by a surgical incision or excision.

19. The method of claim 17 wherein the tissue is traumatized by surgical incision or excision of scar tissue previously formed in the tissue.

20. The method of claim 17 wherein tissue is traumatized by surgical incision or excision of tissue not previously scarred.
